SEQUENCE LISTING (34 sequences)

SEQ ID NO: 1 - protein, 7 aa, Artificial Sequence
  heavy chain variable region (VH) complementarity determining region (CDR), CDR1
GYAFSSS

SEQ ID NO: 2 - protein, 6 aa, Artificial Sequence
  VH CDR2
YPGDED

SEQ ID NO: 3 - protein, 10 aa, Artificial Sequence
  VH CDR3
SLLYGDYLDY

SEQ ID NO: 4 - protein, 10 aa, Artificial Sequence
  light chain variable region (VL) CDR, CDR1
SASSSVSYMH

SEQ ID NO: 5 - protein, 7 aa, Artificial Sequence
  VL CDR2
DTSKLAS

SEQ ID NO: 6 - protein, 9 aa, Artificial Sequence
  VL CDR3
QQWNINPLT

SEQ ID NO: 7 - protein, 119 aa, Artificial Sequence
  CAT19 VH sequence
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 8 - protein, 107 aa, Artificial Sequence
  CAT19 VL sequence
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 9 - protein, 241 aa, Artificial Sequence
  CAT19 VH-VL single-chain variable fragment (scFv) sequence
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 10 - protein, 488 aa, Artificial Sequence
  CAT19 CAR with CD8 stk and 41BB/CD3z endodomain
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 5 aa, Artificial Sequence
  CD22-binding domain VH CDR1
NFAMA

SEQ ID NO: 12 - protein, 17 aa, Artificial Sequence
  CD22-binding domain VH CDR2
SISTGGGNTYYRDSVKG

SEQ ID NO: 13 - protein, 18 aa, Artificial Sequence
  CD22-binding domain VH CDR3
QRNYYDGSYDYEGYTMDA

SEQ ID NO: 14 - protein, 11 aa, Artificial Sequence
  CD22-binding domain VL CDR1
RSSQDIGNYLT

SEQ ID NO: 15 - protein, 7 aa, Artificial Sequence
  CD22-binding domain VL CDR2
GAIKLED

SEQ ID NO: 16 - protein, 7 aa, Artificial Sequence
  CD22-binding domain VL CDR3
LQSIQYP

SEQ ID NO: 17 - protein, 127 aa, Artificial Sequence
  9A8 VH sequence
EVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVSS

SEQ ID NO: 18 - protein, 107 aa, Artificial Sequence
  9A8 VL sequence
DIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIK

SEQ ID NO: 19 - protein, 250 aa, Artificial Sequence
  9A8 scFV sequence
DIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIKRSGGGGSGGGGSGGGGSEVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVS

SEQ ID NO: 20 - protein, 496 aa, Artificial Sequence
  9A8 chimeric antigen receptor (CAR) sequence
METDTLLLWVLLLLVPGSTGDIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIKRSGGGGSGGGGSGGGGSEVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVSSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 8 aa, Artificial Sequence
  BCMA-binding domain VH CDR1
GFIFSDYN

SEQ ID NO: 22 - protein, 8 aa, Artificial Sequence
  BCMA-binding domain VH CDR2
IIYDGSST

SEQ ID NO: 23 - protein, 9 aa, Artificial Sequence
  BCMA-binding domain VH CDR3
ATRPGPFAY

SEQ ID NO: 24 - protein, 11 aa, Artificial Sequence
  BCMA-binding domain VL CDR1
QSLLHSNGNTY

SEQ ID NO: 25 - protein, 3 aa, Artificial Sequence
  BCMA-binding domain VL CDR2
LVS

SEQ ID NO: 26 - protein, 8 aa, Artificial Sequence
  BCMA-binding domain VL CDR3
VHGTHAWT

SEQ ID NO: 27 - protein, 115 aa, Artificial Sequence
  CAR D8 VH sequence
EVQLVESGGGLVQPGRSLKLSCAASGFIFSDYNMAWVRQAPKKGLEWVATIIYDGSSTNHGDSVKGRFTISRDNAKSTLYLQMDSLRSEDTATYYCATRPGPFAYWGQGTLVTVS

SEQ ID NO: 28 - protein, 111 aa, Artificial Sequence
  CAR D8 VL sequence
DVVLTQTPPTLSATIGQSVSISCRSSQSLLHSNGNTYLHWLLQRPGQSPQFLIYLVSGLGSGVPNRFSGSGSGTDFTLKISGVEAEDLGIYYCVHGTHAWTVGGGTKLELK

SEQ ID NO: 29 - protein, 692 aa, Artificial Sequence
  D8 FabCAR sequence
METDTLILWVLLLLVPGSTGDVVLTQTPPTLSATIGQSVSISCRSSQSLLHSNGNTYLHWLLQRPGQSPQFLIYLVSGLGSGVPNRFSGSGSGTDFTLKISGVEAEDLGIYYCVHGTHAWTVGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRAATNFSLLKQAGDVEENPGPMGWSCIILFLVATATGVHSEVQLVESGGGLVQPGRSLKLSCAASGFIFSDYNMAWVRQAPKKGLEWVATIIYDGSSTNHGDSVKGRFTISRDNAKSTLYLQMDSLRSEDTATYYCATRPGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 521 aa, Artificial Sequence
  calcineurin A alpha isoform
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 31 - protein, 170 aa, Artificial Sequence
  calcineurin B, type 1
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 32 - protein, 172 aa, Artificial Sequence
  mutant calcineurin B, CnB30
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNTKLADTQLQQIVDKTIINADKDGDGRISFEEFCAVVGGLDIHKKMVVDV

SEQ ID NO: 33 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 34 - protein, 7 aa, Artificial Sequence
  consensus Tobacco Etch Virus (TEV) cleavage site
ENLYFQS

(19) United States
(12) Patent Application Publication
(10) Pub. No.: US 2023/0338534 A1
(43) Pub. Date: Oct. 26, 2023

(54) METHOD

(71) Applicant: AUTOLUS LIMITED, London (GB)

(72) Inventor: Martin Pulé, London (GB)

(21) Appl. No.: 17/915,737

(22) PCT Filed: Apr. 8, 2021
(86) PCT No.: PCT/GB2021/050863

§ 371 Date: Sep. 29, 2022

(30) Foreign Application Priority Data
Apr. 8, 2020 (GB) 2005216.3

(51) Int. Cl. A61K 39/00

(52) U.S. Cl. CPC A61K 39/4637; A61K 39/4631; A61K 39/46433; A61K 39/4644; A61K 39/46449; A61K 39/4611; A61K 39/464412; A61K 39/464411; A61K 39/4622; A61K 39/464463; A61K 2239/17; A61K 2239/11; A61K 2239/26; A61K 2239/57; A61K 2239/46

(57) ABSTRACT

The present invention provides a method for treating a disease in a subject, which comprises the step of administering to the subject a plurality of cells which express: (a) a chimeric antigen receptor (CAR); and (b) a mutant version of calcineurin A and/or calcineurin B which is resistant to the calcineurin inhibitor. The subject may be receiving or have received treatment with a calcineurin inhibitor. The CAR-expressing cells may be administered prior to, following, simultaneously with or in combination with a calcineurin inhibitor.

### FIELD OF THE INVENTION

The invention relates to methods using cells which co-express a chimeric antigen receptor (CAR) and a mutant version of calcineurin A and/or calcineurin B which is resistant to a calcineurin inhibitor. The invention provides a method for treating a disease in a subject, which comprises the step of administering such cells to a subject. The subject may be receiving or have received treatment with a calcineurin inhibitor. The CAR-expressing cells may be administered prior to, following, simultaneously with or in combination with a calcineurin inhibitor.

### BACKGROUND TO THE INVENTION

### Prevention of Rejection

In solid organ transplants or hematopoietic stem cell transplants (HSCT), mismatches in HLA between recipient and donor can lead to rejection of the organ or graft-vs-host disease (GVHD) respectively. Immunosuppressive drugs can mitigate these outcomes but, due to their broadly inhibitory action against immune cells, they increase the risk of opportunistic infections.

Alloreactive T-cells that recognise mismatches HLA via their T-cell receptor (TCR) are major mediators of rejection and GVHD. CD8+ T cell specificity is dictated by the clonotypic TCR which recognises short antigenic peptides presented on MHC class I molecules. MHC class I molecules are non-covalent heterodimers made up of the membrane-integral, highly polymorphic α-chain and the non-membrane attached non-polymorphic β2 microglobulin (β₂m).

Adoptive cell therapy (ACT) involves administrating disease-relevant immune cells to a subject. For example, where the subject has a cancer, ACT may involve administering immune cells with direct anticancer activity.

ACT using naturally occurring tumour-reactive lymphocytes has mediated durable, complete regressions in patients with melanoma and has also been used in the treatment of epithelial cancers. In addition, the ability to genetically engineer lymphocytes to express conventional T cell receptors (TCRs) or chimeric antigen receptors (CARs) has further extended the successful application of ACT for cancer treatment.

Graft rejection by the host is also a problem for ACT, particularly for CAR-T cells.

CARs are artificial proteins which are typically composed of a targeting domain, a spacer domain, a transmembrane domain and a signaling domain. The targeting domain is typically derived from an scFv which may be murine. While this scFv can be human or humanized and other components individually are derived from self-proteins, the junctions between them can still be immunogenic. For instance, within the scFv there are junctions between the heavy chain and the linker and the linker and the light chain. There is then a junction between the scFv and the spacer domain. If the transmembrane domain is not continuous with the spacer there is a further junction there. Similarly, if the transmembrane domain is not continuous with the amino-terminal portion of the endodomain, there is a further junction there. Finally, most endodomains have at least two components and sometimes more with junctions subsequently between each component.

In addition, CAR T-cells are often engineered with further components. These components include suicide genes (e.g. the HSV-TK enzyme). This enzyme was found to be highly immunogenic and caused a cellular immune depletion of CAR T-cells outside of the context of the profound immunosuppression of haploidentical haematopoietic stem cell transplantation. Other less immunogenic suicide genes may still provide some immunogenicity, as almost every kind of engineered component which involves a fusion between two proteins or use of a xenogeneic protein can be immunogenic.

An important concern in CAR T-cell therapy is the production of antibodies and CTLs against these non-natural components which may result in CAR T-cell rejection. Several studies using murine CAR scFvs have reported that anti-murine scFv CAR antibodies and CTLs are generated after CAR T-cell infusion. The problem of CAR-T cell rejection is particularly acute where the treatment involves multiple rounds of CAR T cell reinfusion.

To date, CAR T-cells have been primarily generated from autologous T-cells. However, in some circumstances, T-cells from an allogeneic donor are used. This can occur if for instance the patient has had an allogeneic haematopoietic stem cell transplant. In this case, harvested T-cells will be allogeneic. Otherwise, a patient may have insufficient T-cells to generate a CAR T-cell product due to chemotherapy induced lymphopenia.

Rejection of allogeneic cells can be due to minor mismatch or major mismatch. Minor mismatch occurs in the setting where allogeneic T-cells are human leukocyte antigen (HLA)-matched to the recipient. In this case, rejection occurs due to minor histocompatibility antigens which are non-HLA differences between individuals which result in presentation of non-self (donor) epitopes/immunogeneic peptides on HLA. In the case where donor and recipient are mismatched, or are only partially matched. T-cell receptors (TCR) on endogenous T-cells of a recipient can interact in a non-specific way with a mismatched HLA and cause rejection consequently. Both minor and major forms of allogeneic rejection are caused by HLA interacting with TCR.

There is thus a need to address the problem of rejection of adoptively transferred immune cells, particularly in allogeneic settings and where the treatment involves repeated dosing of CAR-T cells.

### Preconditioning

ACT has multiple advantages compared with other forms of cancer immunotherapy which rely on the active in vivo development of sufficient numbers of anti-tumour cells with the function necessary to mediate cancer regression. For use in ACT, large numbers of antitumor lymphocytes (up to 10¹¹) can be readily grown in vitro and selected for high-avidity recognition of the tumour, as well as for the effector functions required to mediate cancer regression. In vitro activation allows such cells to be released from the inhibitory factors that exist in vivo. Also, ACT enables the manipulation of the host before cell transfer to provide a favourable microenvironment that better supports antitumor immunity.

In this respect, it has been shown that preconditioning a patient with one or more immunosuppressive chemotherapy drugs prior to T cell infusion can increase the effectiveness of the transplanted T cells. For example, patients may receive cyclophosphamide and fludarabine as preconditioning to decrease immunosuppressive cells prior to T cell infusion. Pre-conditioning patients prior to T cell therapies with cyclophosphamide and fludarabine improves the efficacy of the T cell therapy by reducing the number of endogenous lymphocytes and increasing the serum level of homeostatic cytokines and/or pro-immune factors present in the patient.

Fludarabine is a nucleoside analog which induces cellular cytotoxicity via multiple pathways that ultimately lead to an inhibition of DNA synthesis. A rate-limiting step in this process is the activity of deoxycytidine kinase, which is abundant in lymphocytes, making them susceptible to accumulation of F-ara-ATP, the active metabolite of fludarabine, and hence giving fludarabine particularly potent lymphodepleting properties.

A disadvantage of using fludarabine is the associated myelosuppression which can be profound and sometimes even fatal. Fludarabine also has known associations with neurotoxicity. In a recent clinical trial using CAR-T cells engineered to target the CD19 B-cell antigen to treat acute lymphoblastic leukemia, JCAR-015 (NCT02535364), two patient deaths due to cerebral edema led to trial suspension and fludarabine was suggested as the causative agent.

There is therefore a need for alternative preconditioning regimes, preferably ones which provide a viable alternative to the use of fludarabine.

### The Use of Calcineurin Inhibitors in Transplant and Autoimmunity

Calcineurin inhibitors have been mainstays of immunosuppression in solid organ transplantation since the discovery of cyclosporine in the 1970s. Calcineurin inhibitors are also commonly used in the treatment of autoimmune diseases such as systemic lupus erythematosus and rheumatoid arthritis.

Commonly used calcineurin inhibitors (CNIs) include cyclosporine A (CsA), and tacrolimus (also known as FK506). Cyclosporine is a lipophilic cyclic peptide of 11 amino acids, while tacrolimus is a macrolide antibiotic. Both drugs have been isolated from fungi and possess similar suppressive effects on cell-mediated and humoral immune responses.

Calcineurin is formed by two subunits: A, which is a catalytic subunit (CnA) responsible for its phosphatase activity, and B, a regulatory subunit (CnB) that is responsive to intracellular calcium and regulates CnA activation. As shown in FIG. 1, T cell activation through TCR stimulation elevates intracellular calcium concentration and activates CnB, which unleashes the phosphatase activity of CnA. Activated CnA dephosphorylates cytoplasmic NFATc, a transcription factor, which causes its translocation, along with the activated calcineurin, into the nucleus where it upregulates the expression of multiple cytokines and costimulatory molecules necessary for full activation of T cells. Among NFAT family members, NFAT1, NFAT2, and NFAT4 are involved in the transcriptional activation of genes encoding cytokines, including IL-2 and IL-4, and CD40 ligand. Production of IL-2, in particular, stimulates the growth and differentiation of T cells.

Because of their nature of action, it is not possible to treat a patient who is receiving a CNI, for example following a transplant or for an autoimmune disease, with an adoptive T-cell therapy. There are many settings in which this may otherwise be desirable, for example, for the treatment of cancers which frequently occur in transplant patients such as post-transplant lymphoproliferative disorders (PTLD) or melanomas. Moreover, CAR-T cells themselves may be used for the treatment of autoimmune diseases.

There is therefore a need for an alternative approach to generalised immunosuppression which does not suffer from the drawbacks mentioned above.

### DESCRIPTION OF THE FIGURES

FIG. 1—The role of calcineurin in T cell activation. TCR recognition of the alloantigen (step 1) leads to an increase in the intracellular calcium concentration of T cells (step 2), activating CnB (step 3). Once activated, the CnB unleashes the phosphatase activity of CnA (step 4). Activated CnA dephosphorylates cytoplasmic NFATc (step 5), a transcription factor, allowing for its translocation with activated calcineurin into the nucleus (step 6) where it upregulates the expression of multiple cytokines and costimulatory molecules necessary for full activation of T cells (step 7). Generated IL-2 binds to the IL-2 receptors and induces cell activation and proliferation (step 8).

FIG. 2: Graphs to show the (A) percentage and (B) number of CAR-expressing (RQR8-positive) cells proliferating after 96 hours co-culture with Jurkat KO, Jurkat TRBC1 and Jurkat TRBC2 target cells, in the absence of Tacrolimus

FIG. 3: Graphs to show the (A) percentage and (B) number of CAR-expressing (RQR8-positive) cells proliferating after 96 hours co-culture with Jurkat KO, Jurkat TRBC1 and Jurkat TRBC2 target cells, in the presence of 20 ng/ml of Tacrolimus.

FIG. 4: Graphs to show the number of CAR-expressing (RQR8-positive) cells in each division following co-culture with Jurkat KO, Jurkat TRBC1 and Jurkat TRBC2 target cells, in the absence of Tacrolimus. Proliferation analysis was calculated on single/live/CellTrace Violet-positive cells using FlowJo™ proliferation tool and the CD19 CAR used as the negative control for all the conditions. Cell number in each division is plotted for each CAR+target combination.

FIG. 5: Graphs to show the number of CAR-expressing (RQR8-positive) cells in each division following co-culture with Jurkat KO, Jurkat TRBC1 and Jurkat TRBC2 target cells, in the presence of 20 ng/ml of Tacrolimus. Proliferation analysis was calculated on single/live/CellTrace Violet-positive cells using FlowJo™ proliferation tool and the CD19 CAR used as the negative control for all the conditions. Cell number in each division is plotted for each CAR+target combination.

FIG. 6: Histogram plots showing the proliferation of CAR-expressing (RQR8-positive) cells following co-culture with Jurkat KO, Jurkat TRBC1 and Jurkat TRBC2 target cells, with or without the addition of 20 ng/ml of Tacrolimus. Proliferation analysis was calculated on single/live/CellTrace Violet-positive cells using FlowJo™ proliferation tool and the CD19 CAR used as the negative control for all the conditions. Results are shown using cells from two separate donors.

FIG. 7: Graph showing the cell count of non-transduced cells (NT) and TRBC2 CAR-expressing (RQR8-positive) cells before (day 0) and after (day 4) co-culture with TRBC2 targets with or without the addition of 20 ng/ml of Tacrolimus.

FIG. 8: Graph showing the percentage of TRBC2 CAR-expressing (RQR8-positive) cells before (day 0) and after (day 4) co-culture with TRBC2 targets with or without the addition of 20 ng/ml of Tacrolimus.

### SUMMARY OF ASPECTS OF THE INVENTION

The present invention relates to a new approach which facilitates the simultaneous use of calcineurin inhibitors (CNIs) and adoptive T-cell therapy, such as the use of CAR-T cells.

This approach has a variety of different clinical applications, for example:
it enables a patient to be preconditioned with a CNI prior to adoptive cell therapy, thereby potentially avoiding the use of alternative preconditioning agents such as fludarabine which may have undesirable side effects;
it enables CNIs to be used to prevent rejection of the grafted T cells by the host, which is particularly useful for allogeneic grafted T cells and therapies involving repeated doses of engineered T cells; and
it enables adoptive T-cell therapies to be used on a patient who is already being treated with a CNI, for example following transplant or to treat and/or prevent autoimmune disease.

Thus, in a first aspect, the invention provides a method for treating and/or preventing a disease in a subject who is receiving or has received treatment with a calcineurin inhibitor, which method comprises the step of administering to the subject a plurality of cells which express:
(a) a chimeric antigen receptor (CAR); and
(b) a mutant version of calcineurin A and/or calcineurin B which is resistant to the calcineurin inhibitor.

The invention also provides a plurality of cells for use in a method of treating and/or preventing a disease in a subject who is receiving or has received treatment with a calcineurin inhibitor, wherein the plurality of cells express:
(a) a chimeric antigen receptor (CAR); and
(b) a mutant version of calcineurin A and/or calcineurin B which is resistant to the calcineurin inhibitor.

There is also provided the use of a plurality of cells in the manufacture of a pharmaceutical composition for use in the a method of treating and/or preventing a disease in a subject who is receiving or has received treatment with a calcineurin inhibitor, wherein the plurality of cells express:
(a) a chimeric antigen receptor (CAR); and
(b) a mutant version of calcineurin A and/or calcineurin B which is resistant to the calcineurin inhibitor.

The subject may be receiving or have received treatment with a calcineurin inhibitor in order to treat and/or prevent an autoimmune disease. For such patients, the CAR may bind an autoantigen associated with the autoimmune disease.

Alternatively, the subject may be post-transplant and be receiving or have received treatment with a calcineurin inhibitor in order to prevent transplant rejection. For such patients the CAR may binds a cancer antigen. The method may be to treat and/or prevent a transplant-associated cancer such as post-transplant lymphoproliferative disorder (PTLD) or a melanoma.

In a second aspect, the invention provides a method for treating a disease in a subject, which comprises the following steps:
(i) preconditioning a patient with a calcineurin inhibitor; and
(ii) administering to the subject a plurality of cells which express:
   (a) a chimeric antigen receptor (CAR); and
   (b) a mutant version of calcineurin A and/or calcineurin B which is resistant to the calcineurin inhibitor.

The invention also provides a plurality of cells for use in a method of treating a disease in a subject, wherein the plurality of cells express:
(a) a chimeric antigen receptor (CAR); and
(b) a mutant version of calcineurin A and/or calcineurin B which is resistant to a calcineurin inhibitor, and wherein the method involves
   (i) preconditioning a patient with the calcineurin inhibitor; and
   (ii) administering the plurality of cells to the subject.

There is also provided the use of a plurality of cells in the manufacture of a pharmaceutical composition for use in a method of treating a disease in a subject, wherein the plurality of cells express:
(a) a chimeric antigen receptor (CAR); and
(b) a mutant version of calcineurin A and/or calcineurin B which is resistant to a calcineurin inhibitor, and wherein the method involves
   (i) preconditioning a patient with the calcineurin inhibitor; and
   (ii) administering the plurality of cells to the subject.

The CAR-expressing cells may be allogeneic.

The method may involve administering a plurality of doses of the CAR-expressing cells to the subject.

The calcineurin inhibitor may be selected from cyclosporine A (CsA), and tacrolimus.

The CAR may bind to a B-cell antigen or a plasma cell antigen. For example, the CAR may bind CD19, CD20, CD22 or BCMA.

The CAR may bind CD19 and have a CD19-binding domain which comprises a) a heavy chain variable region (VH) having complementarity determining regions (CDRs) with the following sequences:
CDR1—GYAFSSS (SEQ ID No. 1);
CDR2—YPGDED (SEQ ID No. 2)
CDR3—SLLYGDYLDY (SEQ ID No. 3); and

b) a light chain variable region (VL) having CDRs with the following sequences:
CDR1—SASSSVSYMH (SEQ ID No. 4);
CDR2—DTSKLAS (SEQ ID No. 5)
CDR3—QQWNINPLT (SEQ ID No. 6).

The cells may express one of the following mutant versions of calcineurin A and/or calcineurin B:
calcineurin A comprising mutations T351E and L354A with reference to the shown as SEQ ID No. 30;
calcineurin A comprising mutations V314R and Y341F and with reference to shown as SEQ ID No 30; or
calcineurin B comprising mutation L124T and K-125-LA-Ins with reference to shown as SEQ ID No. 31.

For example, the cells may express mutant calcineurin B having the sequence shown as SEQ ID No. 32.

Further aspects of the invention are summarised in the following numbered paragraphs.

1. A cell which expresses a chimeric antigen receptor (CAR) and a mutant version of calcineurin A and/or calcineurin B which is resistant to a calcineurin inhibitor.

2. A cell according to paragraph 1, which expresses:
calcineurin A comprising mutations T351E and L354A with reference to the shown as SEQ ID No. 30;
calcineurin A comprising mutations V314R and Y341F and with reference to shown as SEQ ID No 30; or
calcineurin B comprising mutation L124T and K-125-LA-Ins with reference to shown as SEQ ID No. 31.

3. A cell according to paragraph 1 or 2 which expresses mutant calcineurin B having the sequence shown as SEQ ID No. 32.

4. A cell according to any preceding paragraph wherein the CAR binds a B-cell antigen or a plasma cell antigen.

5. A cell according to paragraph 4, wherein the CAR binds CD19, CD20, CD22 or BCMA

6. A cell according to paragraph 5, wherein the CAR targets CD19.

7. A cell according to paragraph 6, wherein the CAR comprises a CD19-binding domain which comprises a) a heavy chain variable region (VH) having complementarity determining regions (CDRs) with the following sequences:
CDR1—GYAFSSS (SEQ ID No. 1);
CDR2—YPGDED (SEQ ID No. 2)
CDR3—SLLYGDYLDY (SEQ ID No. 3); and

b) a light chain variable region (VL) having CDRs with the following sequences:
CDR1—SASSSVSYMH (SEQ ID No. 4);
CDR2—DTSKLAS (SEQ ID No. 5)
CDR3—QQWNINPLT (SEQ ID No. 6).

8. A nucleic acid construct which comprises:
(i) a first nucleic acid sequence which encodes a CAR; and
(ii) a second nucleic acid sequence which encodes a mutant version of calcineurin A and/or calcineurin B which is resistant to a calcineurin inhibitor as defined in any of paragraphs 1 to 3.

9. A vector comprising a nucleic acid construct according to paragraph 8.

10. A kit of vectors comprising:
(i) a first vector comprising a nucleic acid sequence which encodes a CAR; and
(ii) a second vector comprising a nucleic acid sequence which encodes a mutant version of calcineurin A and/or calcineurin B which is resistant to a calcineurin inhibitor.

11. A pharmaceutical composition comprising a plurality of cells according to any of paragraphs 1 to 7.

12. A pharmaceutical composition according to paragraph 11 which also comprises a calcineurin inhibitor.

13. A pharmaceutical composition according to paragraph 12, wherein the calcineurin inhibitor is selected from cyclosporine A (CsA), and tacrolimus.

14. A method for treating and/or preventing a disease, which comprises the step of administering a pharmaceutical composition according to any of paragraphs 11 to 13 to a subject.

15. A method according to paragraph 14, which comprises the following steps:
(i) administering a pharmaceutical composition according to any of paragraphs 11 to 13 to a subject; and
(ii) subsequently administering a calcineurin inhibitor to the subject.

16. A method according to paragraph 15, wherein step (ii) involves regular administration of the calcineurin inhibitor to the subject for a period of about 6 weeks following administration of the cells.

17. A method according to paragraph 16, wherein the calcineurin inhibitor is administered orally to the subject.

18. A method for treating a disease according to any of paragraphs 14 to 17, wherein the subject has cancer or an autoimmune disease.

19. A method according to any of paragraphs 14 to 18, wherein the cells administered to the subject are allogeneic.

20. A pharmaceutical composition according to any of paragraphs 11 to 13 for use in treating a disease.

21. The use of a plurality of cells according to any of paragraphs 1 to 7 in the manufacture of a medicament for the treatment of a disease.

22. A method for making a cell according to any of paragraphs 1 to 7, which comprises the step of introducing: a nucleic acid construct according to paragraph 6, a vector according to paragraph 7 or a kit of vectors according to paragraph 9, into the cell in vitro.

23. A method according to paragraph 26, which comprises the following steps:
(i) obtaining a population of cells;
(ii) transducing or transfecting the cells with a nucleic acid construct according to paragraph 8, a vector according to paragraph 9 or a kit of vectors according to paragraph 10, into the cell;
(iii) adding a calcineurin inhibitor to the cell population from step (ii) in order to enrich for transduced/transfected cells.

### DETAILED DESCRIPTION

The present invention relates to cells which express one or more chimeric antigen receptors (CARs).

### Chimeric Antigen Receptor (CAR)

Classical CARs are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site (such as a Fab) or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 4-1BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on.

Thus, the CAR directs the specificity and cytotoxicity of the T cell towards cells expressing the targeted antigen.

### Antigen Binding Domain

The antigen-binding domain is the portion of a CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a wild-type ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The CAR may bind a B-cell antigen or a plasma cell antigen. For example, the CAR may bind CD19, CD20, CD22 or BCMA

A CD19-targeting CAR is described in WO2016/139487.

This CAR comprises a CD19-binding domain which comprises a) a heavy chain variable region (VH) having complementarity determining regions (CDRs) with the following sequences:
CDR1—GYAFSSS (SEQ ID No. 1);
CDR2—YPGDED (SEQ ID No. 2);
CDR3—SLLYGDYLDY (SEQ ID No. 3); and

b) a light chain variable region (VL) having CDRs with the following sequences:
CDR1—SASSSVSYMH (SEQ ID No. 4);
CDR2—DTSKLAS (SEQ ID No. 5);
CDR3—QQWNINPLT (SEQ ID No. 6).

The CDRs may be in the format of a single-chain variable fragment (scFv), which is a fusion protein of the heavy variable region (VH) and light chain variable region (VL) of an antibody, connected with a short linker peptide of ten to about 25 amino acids. The scFv may be in the orientation VH-VL, i.e. the VH is at the amino-terminus of the CAR molecule and the VL domain is linked to the spacer and, in turn the transmembrane domain and endodomain.

The CDRs may be grafted on to the framework of a human antibody or scFv.

The CAR may comprise the following
VH sequence:
CAT19 VH sequence
SEQ ID No. 7
QVQLQQSGPELVKPGASVKISCKASGYAFSSSWMNWVKQR
PGKGLEWIGRIYPGDEDTNYSGKFKDKATLTADKSSTTAY
MQLSSLTSEDSAVYFCARSLLYGDYLDYWGQGTTLTVSS
The CAR may comprise the following
VL sequence:
CAT19 VL sequence
SEQ ID No 8
QIVLTQSPAIMSASPGEKVTMTCSASSSVSYMHWYQQKSG
TSPKRWIYDTSKLASGVPDRFSGSGSGTSYFLTINNMEAE
DAATYYCQQWNINPLTFGAGTKLELKR
The CAR may comprise the following
scFv sequence:
CAT19 VH-VL scFv sequence
SEQ ID No 9
QVQLQQSGPELVKPGASVKISCKASGYAFSSSWMNWWKQR
PGKGLEWIGRIYPGDEDTNYSGKFKDKATLTADKSSTTAY
MQLSSLTSEDSAVYFCARSLLYGDYLDYWGQGTTLTVSSG
GGGSGGGGSGGGGSQIVLTQSPAIMSASPGEKVTMTCSAS
SSVSYMHWYQQKSGTSPKRWIYDTSKLASGVPDRFSGSGS
GTSYFLTINNMEAEDAATYYCQQWNINPLTFGAGTKLELK
R

The CAR of the invention may comprise a variant of the sequence shown as SEQ ID No. 7, 8 or 9 having at least 80, 85, 90, 95, 98 or 99% sequence identity, provided that the variant sequence retain the capacity to bind CD19 (when in conjunction with a complementary VL or VH domain, if appropriate).

The percentage identity between two polypeptide sequences may be readily determined by programs such as BLAST which is freely available at http://blast.ncbi.nlm.nih.gov.

The CAR may comprise the sequence shown as SEQ ID No. 10, which comprises:
T-cell receptor beta chain V region signal peptide (underlined)
The CAT19 ScFv sequence (shown above as SEQ ID No. 19)
A human CD8 stalk spacer sequence (shown in bold)
The human CD8 stalk TM sequence (shown in italics)
A 41BB endodomain sequence (shown underlined and italics)
A CD3z endodomain sequence (shown in bold and italics)

CAT19 CAR with CD8 stk and 41BB/CD3z endodomain
SEQ ID No. 10
MGTSLLCWMALCLLGADHADAQVQLQQSGPELVKPGASV
KISCKASGYAFSSSWMNWWKQRPGKGLEWIGRIYPGDEDT
NYSGKFKDKATLTADKSSTTAYMQLSSLTSEDSAVYFCAR
SLLYGDYLDYWGQGTTLTVSSGGGGSGGGGSGGGGSQIVL
TQSPAIMSASPGEKVTMTCSASSSVSYMHWYQQKSGTSPK
RWIYDTSKLASGVPDRFSGSGSGTSYFLTINNMEAEDAAT
YYCQQWNINPLTFGAGTKLELKRSDPTTTPAPRPPTPAPT
IASQPLSLRPEACRPAAGGAVHTRGLDFACDIYIWAPLA
GTCGVLLLSLVITLYCKRGRKKLLYIFKQPFMRPVQTTQE
EDGCSCRFPEEEEGGCELRVKFSRSADAPAYQQGQNQLYN
ELNLGRREEYDVLDKRRGRDPEMGGKPRRKNPQEGLYNEL
QKDKMAEAYSEIGMKGERRRGKGHDGLYQGLSTATKDTYD
ALHMQALPPR

The CAR of the invention may comprise a variant of the sequence shown as SEQ ID No. 10 having at least 80, 85, 90, 95, 98 or 99% sequence identity, provided that the variant sequence retain the capacity to bind CD19 and induce a T-cell activation signal.

A CD22-targeting CAR is described in WO2019/220109.

This CAR comprises a CD22-binding domain which comprises a) a heavy chain variable region (VH) having complementarity determining regions (CDRs) with the following sequences:
CDR1—NFAMA (SEQ ID No. 11)
CDR2—SISTGGGNTYYRDSVKG (SEQ ID No. 12)
CDR3—QRNYYDGSYDYEGYTMDA (SEQ ID No. 13); and

b) a light chain variable region (VL) having complementarity determining regions (CDRs) with the following sequences:
CDR1—RSSQDIGNYLT (SEQ ID No. 14)
CDR2—GAIKLED (SEQ ID No. 15)
CDR3—LQSIQYP (SEQ ID No. 16).

The CAR may comprise the following VH sequence:
9A8 VH sequence
SEQ ID No. 17
EVQLVESGGGLVQPGRSLKLSCAASGFTFSNFAMAWRQPP
TKGLEWASISTGGGNTYYRDSVKGRFTISRDDAKNTQYLQ
MDSLRSEDTATYYCARQRNYYDGSYDYEGYTMDAWGQGTS
VTVSS
The CAR may comprise the following VL sequence:
9A8 VL sequence
SEQ ID No 18
DIQMTQSPSSLSASLGDRVTITCRSSQDIGNYLTWFQQKV
GRSPRRMIYGAIKLEDGVPSRFSGSRSGSDYSLTISSLES
EDVADYQCLQSIQYPFTFGSGTKLEIK
9A8 scFV sequence
SEQ ID No 19
DIQMTQSPSSLSASLGDRVTITCRSSQDIGNYLTWFQQKV
GRSPRRMIYGAIKLEDGVPSRFSGSRSGSDYSLTISSLES
EDVADYQCLQSIQYPFTFGSGTKLEIKRSGGGGSGGGGSG
GGGSEVQLVESGGGLVQPGRSLKLSCAASGFTFSNFAMAW
RQPPTKGLEWWASISTGGGNTYYRDSVKGRFTISRDDAKN
TQYLQMDSLRSEDTATYYCARQRNYYDGSYDYEGYTMDAW
GQGTSVTVS

The CAR may comprise a variant of the sequence shown as SEQ ID No. 17 to 19 having at least 80, 85, 90, 95, 98 or 99% sequence identity, provided that the variant sequence retain the capacity to bind CD22 (when in conjunction with a complementary VL or VH domain, if appropriate).

The CAR may comprise the sequence shown as SEQ ID No. 20, which comprises:
Murine Ig kappa signal peptide (underlined)
ScFv sequence (shown above as SEQ ID No. 19)
human CD8a stalk spacer sequence (shown in bold)
human CD8a stalk TM sequence (shown in italics)
A 41BB endodomain sequence (shown underlined and italics)
A CD3z endodomain sequence (shown in bold and italics)

9A8 CAR
SEQ ID No. 20
METDTLLLWLLLLVPGSTGDIQMTQSPSSLSASLGDRVTI
TCRSSQDIGNYLTWFQQKVGRSPRRMIYGAIKLEDGVPSR
FSGSRSGSDYSLTISSLESEDVADYQCLQSIQYPFTFGSG
TKLEIKRSGGGGSGGGGSGGGGSEVQLVESGGGLVQPGRS
LKLSCAASGFTFSNFAMAWRQPPTKGLEWVASISTGGGNT
YYRDSVKGRFTISRDDAKNTQYLQMDSLRSEDTATYYCAR
QRNYYDGSYDYEGYTMDAWGQGTSVTVSSDPTTTPAPRPP
TPAPTIASQPLSLRPEACRPAAGGAVHTRGLDFACDIYIW
APLAGTCGVLLLSLVITLYCKRGRKKLLYIFKQPFMRPVQ
TTQEEDGCSCRFPEEEEGGCELRVKFSRSADAPAYQQGQN
QLYNELNLGRREEYDVLDKRRGRDPEMGGKPRRKNPQEGL
YNELQKDKMAEAYSEIGMKGERRRGKGHDGLYQGLSTATK
DTYDALHMQALPPR

The CAR may comprise a variant of the sequence shown as SEQ ID No. 20 having at least 80, 85, 90, 95, 98 or 99% sequence identity, provided that the variant sequence retain the capacity to bind CD22 and induce a T-cell activation signal.

A BCMA-targeting CAR is described in WO2020/065330.

This CAR comprises a BCMA-binding domain which comprises a) a heavy chain variable region (VH) having complementarity determining regions (CDRs) with the following sequences:
CDR1—GFIFSDYN (SEQ ID No. 21)
CDR2—IIYDGSST (SEQ ID No. 22)
CDR3—ATRPGPFAY (SEQ ID No. 23); and

b) a light chain variable region (VL) having complementarity determining regions (CDRs) with the following sequences:
CDR1—QSLLHSNGNTY (SEQ ID No. 24)
CDR2—LVS (SEQ ID No. 25)
CDR3—VHGTHAWT (SEQ ID No. 26)

SEQ ID No. 27
D8 VH sequence
EVQLVESGGGLVQPGRSLKLSCAASGFIFSDYNMAWWRQA
PKKGLEWVATIIYDGSSTNHGDSVKGRFTISRDNAKSTLY
LQMDSLRSEDTATYYCATRPGPFAYWGQGTLVTVS
The CAR may comprise the following VL sequence:
D8 VL sequence
SEQ ID No 28
DVVLTQTPPTLSATIGQSVSISCRSSQSLLHSNGNTYLHW
LLQRPGQSPQFLIYLVSGLGSGVPNRFSGSGSGTDFTLKI
SGVEAEDLGIYYCVHGTHAWTVGGGTKLELK

The CAR may comprise a variant of the sequence shown as SEQ ID No. 27 and/or 28 having at least 80, 85, 90, 95, 98 or 99% sequence identity, provided that the variant sequence retain the capacity to bind CD22 (when in conjunction with a complementary VL or VH domain, if appropriate).

The ant-BCMA CAR may be a FabCAR. The CAR may comprise the sequence shown as SEQ ID No. 29, which comprises:
Murine Ig kappa signal peptide (underlined)
anti-BCMA VL sequence (shown above as SEQ ID No.28)
Human Ig kappa constant region (shown in italics)
P2A self-cleaving peptide (shown in bold)
Murine Ig heavy chain V region 102 signal peptide (shown in grey and underlined)
anti-BCMA VH sequence (shown above as SEQ ID No.27; in grey)
Human IgG1-CH1 domain (shown in grey italics)
Human IgG1-Hinge (in bold and underlined)
Linker (in italics and underlined)
Human CD28 TM sequence (in bold and italics)
A 41BB endodomain sequence (in grey underlined and italics)
A CD3z endodomain sequence (in grey bold and italics)

SEQ ID No. 29
D8 FabCAR
METDTLILWWLLLLVPGSTGDVVLTQTPPTLSATIGQSVSISCRSSQSL
LHSNGNTYLHWLLQRPGQSPQFLIYLVSGLGSGVPNRFSGSGSGTDFTL
KISGVEAEDLGIYYCVHGTHAWTVGGGTKLELKRTVAAPSVFIFPPSDE
QLKSGTASVVCLLNNFYPREAKVQWKVDNALQSGNSQESVTEQDSKDST
YSLSSTLTLSKADYEKHKVYACEVTHQGLSSPVTKSFNRGECRAATNFS
LLKQAGDVEENPGP

[symbol]

[symbol]

[symbol]

[symbol]

[symbol]

[symbol]

[symbol]

[symbol]

[symbol]

[symbol]

[symbol]

[symbol]

[symbol]

[symbol]

EPKSCDKT
HTCPPCPKDPKFWVLVVVGGVLACYSLLVTVAFI
IFWV

[symbol]

[symbol]

[symbol]

[symbol]

[symbol]

[symbol]

[symbol]

[symbol]

[symbol]

[symbol]

The CAR comprise a variant of the sequence shown as SEQ ID No. 29 having at least 80, 85, 90, 95, 98 or 99% sequence identity, provided that the variant sequence retain the capacity to bind BCMA and induce a T-cell activation signal.

Where the method of the invention is for the treatment of an autoimmune disease, the CAR may target an autoantigen. For example, the CAR may target . . . .

The target antigen may be expressed on a solid cancer. For example, the target may be PSMA for the treatment of prostate cancer.

The CAR-expressing cell may target a cell which is not a T-cell, such as a B-cell, plasma cell or cancerous cell of non-T-cell origin such a malignant epithelial cell. The CAR target antigen may not form part of the T-cell receptor complex. The target antigen may be an antigen other than TRBC1 or TRBC2.

### Transmembrane Domain

The transmembrane domain is the sequence of a CAR that spans the membrane. It may comprise a hydrophobic alpha helix. The transmembrane domain may be derived from any transmembrane protein such as from the melanocyte protein Tyrp1, or from CD8 or CD28.

### CAR Signal Peptide

The CAR for use in the present invention may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

### Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

### Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41BB, CD27, GITR etc). For example, chimeric CD28 and OX40 can be used with CD3-Zeta to transmit a proliferative/survival signal, or all three can be used together.

The endodomain may comprise:
(i) an ITAM-containing endodomain, such as the endodomain from CD3 zeta; and/or
(ii) a co-stimulatory domain, such as the endodomain from CD28 or ICOS; and/or
(iii) a domain which transmits a survival signal, for example a TNF receptor family endodomain such as OX-40, 4-1BB, CD27 or GITR.

A number of systems have been described in which the antigen recognition portion is on a separate molecule from the signal transmission portion, such as those described in WO015/150771; WO2016/124930 and WO2016/030691. The CAR of the present invention may therefore comprise an antigen-binding component comprising an antigen-binding domain and a transmembrane domain; which is capable of interacting with a separate intracellular signalling component comprising a signalling domain. The vector of the invention may express a CAR signalling system comprising such an antigen-binding component and intracellular signalling component.

### Calcineurin Mutants

The cells of the present invention co-express a CAR and a mutant version of calcineurin A and/or calcineurin B which is resistant to a calcineurin inhibitor.

Calcineurin (CaN) is a calcium and calmodulin dependent serine/threonine protein phosphatase involved in T-cell activation. Calcineurin is a heterodimer of a 61-kD calmodulin-binding catalytic subunit, calcineurin A (CnA) and a 19-kD Ca²⁺-binding regulatory subunit, calcineurin B (CnB).

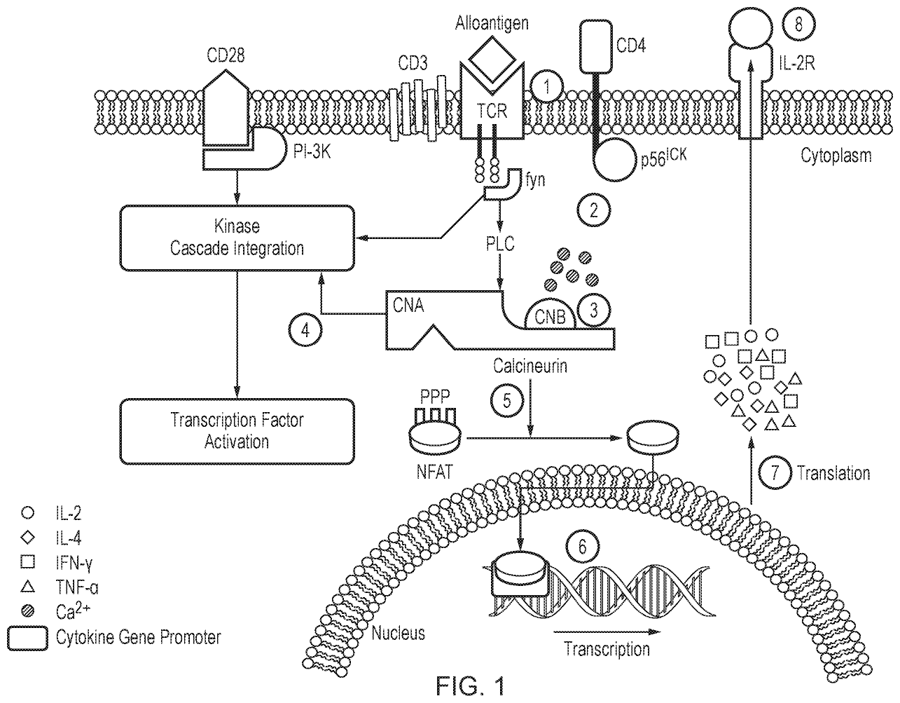

The role of calcineurin in T-cell activation is shown in FIG. 1. Recognition of an antigen by the T-cell receptor (TCR) leads to an increase in the intracellular calcium concentration of T cells activating CnB. Once activated, the CnB unleashes the phosphatase activity of CnA. Activated CnA dephosphorylates cytoplasmic NFATc, a transcription factor, allowing for its translocation with activated calcineurin into the nucleus where it upregulates the expression of multiple cytokines (including IL2) and costimulatory molecules necessary for full activation of T cells.

Calcineurin is the target of a class of drugs called calcineurin inhibitors, which include cyclosporin, voclosporin, pimecrolimus and tacrolimus.

The calcineurin inhibitor may be cyclosporine A (CsA) or tacrolimus (also known as FK506). Cyclosporine is a lipophilic cyclic peptide of 11 amino acids, while tacrolimus is a macrolide antibiotic. Both drugs have been isolated from fungi and possess similar suppressive effects on cell-mediated and humoral immune responses. CNIs bind intracellular proteins called immunophilins: cyclophilins in the case of cyclosporine, and the FK-binding proteins in the case of tacrolimus. The drug-receptor complex specifically and competitively binds to and inhibits calcineurin, leading to reduced transcriptional activation of cytokine genes and reduction in proliferation of T lymphocytes.

Cyclosporine and tacrolimus are the most commonly used CNIs in transplant recipients. Cyclosporine was initially approved in 1983 by the U.S. Food and Drug Administration (FDA) for immunosuppression following organ transplantation, and in 1995 a microemulsion formulation of cyclosporine (associated with better bioavailability and more consistent absorption) was approved. Cyclosporine formulations are usually administered twice daily. Tacrolimus received FDA approval in 1994 for liver transplant recipients, and in 1997 for kidney transplants. Tacrolimus is usually administered twice daily, but recently became available as an extended release once-daily formulation. FDA-approved generic equivalents are available for tacrolimus immediate release formulations, as well as modified and unmodified cyclosporine.

The cell of the present invention may express calcineurin A and/or B comprising one or more mutations which increases its resistance to one or more immune suppressive drugs. For example, the cell may comprise one or more mutations which renders the cell resistant to tacrolimus and/or cyclosporin.

The cell may comprise a nucleic acid sequence encoding calcineurin (CN) with one or more mutations. Brewin et al (2009; Blood 114: 4792-4803) describe various calcineurin mutants which render cytotoxic T lymphocytes resistant to tacrolimus and/or cyclosporin.

As explained above, calcineurin is a heterodimer of a 61-kD calmodulin-binding catalytic subunit, calcineurin A and a 19-kD Ca2+-binding regulatory subunit, calcineurin B. There are three isozymes of the catalytic subunit, each encoded by a separate gene (PPP3CA, PPP3CB, and PPP3CC) and two isoforms of the regulatory, also encoded by separate genes (PPP3R1, PPP3R2). The amino acid sequences for all of the polypeptides encoded by these genes are available from Uniprot, with the following accession numbers: PPP3CA: Q08209; PPP3CB: P16298; PPP3CC: P48454; PPP3R1: P63098; and PPP3R2: Q96LZ3.

The amino acid sequence for calcineurin A, alpha isoform is shown below as SEQ ID No. 30.

(calcineurin A)
SEQ ID No. 30
MSEPKAIDPKLSTTDRVVKAVPFPPSHRLTAKEVFDNDGK
PRVDILKAHLMKEGRLEESVALRIITEGASILRQEKNLLD
IDAPVTVCGDIHGQFFDLMKLFEVGGSPANTRYLFLGDYV
DRGYFSIECVLYLWALKILYPKTLFLLRGNHECRHLTEYF
TFKQECKIKYSERVYDACMDAFDCLPLAALMNQQFLCVHG
GLSPEINTLDDIRKLDRFKEPPAYGPMCDILWSDPLEDFG
NEKTQEHFTHNTVRGCSYFYSYPAVCEFLQHNNLLSILRA
HEAQDAGYRMYRKSQTTGFPSLITIFSAPNYLDVYNNKAA
VLKYENNVMNIRQFNCSPHPYWLPNFMDVFTWSLPFVGEK
VTEMLVNVLNICSDDELGSEEDGFDGATAAARKEVIRNKI
RAIGKMARVFSVLREESESVLTLKGLTPTGMLPSGVLSGG
KQTLQSATVEAIEADEAIKGFSPQHKITSFEEAKGLDRIN
ERMPPRRDAMPSDANLNSINKALTSETNGTDSNGSNSSNI
Q

Mutant calcineurin A may comprise a mutation at one or more of the following positions with reference to SEQ ID No. 30: V314; Y341; M347; T351; W352; S353; L354; F356; and K360.

Mutant calcineurin A may comprise one or more of the following substitution mutations with reference to SEQ ID No. 30:
V314K, V314R or V314F;
Y341F;
M347W, M347R or M347E;
T351E;
W352A, W352C or W352E;
S353H or S353N;
L354A;
F356A; and
K360A or K360F.

Mutant calcineurin A may comprise one or more of the following mutation combinations with reference to SEQ ID No. 30:
L354A and K360A;
L354A and K360F;
T351E and K360F;
W352A and S353H;
T351E and L354A;
W352C and K360F;
W352C; L354A and K360F;
V314K and Y341F; and
V314R and Y341F.

The amino acid sequence for calcineurin B, type 1 is shown below as SEQ ID No. 31

(calcineurin B)
SEQ ID No. 31
MGNEASYPLEMCSHFDADEIKRLGKRFKKLDLDNSGSLSV
EEFMSLPELQQNPLVQRVIDIFDTDGNGEVDFKEFIEGVS
QFSVKGDKEQKLRFAFRIYDMDKDGYISNGELFQVLKMMV
GNNLKDTQLQQIVDKTIINADKDGDGRISFEEFCAVVGGL
DIHKKMVVDV

Mutant calcineurin B may comprise a mutation at one or more of the following positions with reference to SEQ ID No. 31: Q51; L116; M119; V120; G121; N122; N123; L124; K125; and K165.

Mutant calcineurin B may comprise one or more of the following substitution and optionally insertion mutations with reference to SEQ ID No. 31:
Q51S;
L116R or L116Y;
M119A, M119W or M119-F-Ins;
V120L, V120S, V120D or V120F;
G121-LF-Ins;
N122A, N122H, N122F or N122S;
N123H, N123R, N123F, N123K or N123W;
L124T;
K125A, K125E, K125W, K125-LA-Ins, K125-VQ-Ins or K125-IE-Ins; and
K165Q.

Mutant calcineurin B may comprise one or more of the following mutation combinations with reference to SEQ ID No. 31:
V120S and L124T;
V120D and L124T;
N123W and K125-LA-Ins;
L124T and K125-LA-Ins;
V120D and K125-LA-Ins; and
M119-F-Ins and G121-LF-Ins.

In particular, mutant calcineurin B may comprise the following mutation combination with reference to SEQ ID No. 31: L124T and K125-LA-Ins. This is the module known as “CnB30” described in the Examples section. The CnB30 has the amino acid shown as SEQ ID No. 32.

(CnB30)
SEQ ID No. 32
MGNEASYPLEMCSHFDADEIKRLGKRFKKLDLDNSGSLSV
EEFMSLPELQQNPLVQRVIDIFDTDGNGEVDFKEFIEGVS
QFSVKGDKEQKLRFAFRIYDMDKDGYISNGELFQVLKMMV
GNNTKLADTQLQQIVDKTIINADKDGDGRISFEEFCAVVG
GLDIHKKMVVDV

In the study described in Brewin et al 2009 (as above), the following CNa mutants showed resistance to FK506:
L354A and K360F;
W352A;
W352C;
T351E and L354A;
M347W; and
M347E.

The following CNa mutants showed resistance to cyclosporin A:
V314K;
V314R;
Y341F;
V314K and Y341F; and
V314R and Y341F.

The following CNb mutants showed resistance to FK506:
N123W;
K125-VQ-Ins;
K125-IE-Ins;
K-125-LA-Ins; and
L124T and K-125-LA-Ins.

The following CNb mutants showed resistance to cyclosporin A:
K125-VQ-Ins;
K125-IE-Ins;
K-125-LA-Ins;
V120S and L124T; and
L124T and K-125-LA-Ins.

In particular, it is reported in Brewin et al 2009 (as above) that:
the combination mutation T351E and L354A in CNa confers resistance to CsA but not FK506;
the combination mutation V314R and Y341F in CNa confers resistance to FK506 but not CsA; and
the combination mutation L124T and K-125-LA-Ins in CNb renders CTLs resistant to both calcineurin inhibitors.

The cell of the present invention may express a variant calcineurin A comprising one or more mutations in the CNa amino acid sequence and/or a variant calcineurin B comprising one or more mutations in the CNb amino acid sequence, which increases resistance of the effector immune cell to one or more calcineurin inhibitors.

In particular, the cell may express a variant calcineurin A and/or a variant calcineurin B as listed above which confers resistance to cyclosporin A and/or tacrolimus (FK506).

### Nucleic Acid Construct

The invention provides a nucleic acid construct which comprises:
(i) a first nucleic acid sequence which encodes a CAR; and
(ii) a second nucleic acid sequence which encodes a mutant version of calcineurin A and/or calcineurin B which is resistant to a calcineurin inhibitor as described above.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed. Suitably, the polynucleotides of the present invention are codon optimised to enable expression in a mammalian cell, in particular an immune effector cell as described herein.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence or amino acid sequence includes any substitution of, variation of, modification of, replacement of, deletion of or addition of one (or more) nucleic acid(s) from or to the sequence.

The nucleic acid construct may have the general structure:
CAR-coexpr-CnM; or
CnM-coexpr-CAR
   in which:
“CAR” is a nucleic acid sequence encoding a CAR;
“coexpr” is a nucleic acid sequence enabling co-expression og the CAR and calcineurin mutant as separate polypeptdes; and
“CnM” is a nucleic acid sequence which encodes a mutant version of calcineurin A and/or calcineurin B which is resistant to a calcineurin inhibitor.

The co-expression site may be a cleavage site. The cleavage site may be any sequence which enables the two polypeptides to become separated. The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site. Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide. A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above.

The co-expression sequence may be an internal ribosome entry sequence (IRES). The co-expressing sequence may be an internal promoter.

The present invention also provides a kit comprising of nucleic acid sequences which comprises:
a first polynucleotide which encodes a chimeric antigen receptor (CAR); and
a second polynucleotide which encodes which encodes a mutant version of calcineurin A and/or calcineurin B which is resistant to a calcineurin inhibitor.

### Vector

The present invention also provides a vector which comprises a nucleic acid construct(s) of the invention.

The invention also provides a kit of vectors comprising:
(i) a first vector comprising a nucleic acid sequence which encodes a CAR; and
(ii) a second vector comprising a nucleic acid sequence which encodes a mutant version of calcineurin A and/or calcineurin B which is resistant to a calcineurin inhibitor.

Such a vector or kit of vectors may be used to introduce the nucleic acid sequence(s) or construct(s) into a host cell so that it co-expresses a CAR together with a mutant version of calcineurin A and/or calcineurin B.

The vector may, for example, be a plasmid or a viral vector, such as a retroviral vector or a lentiviral vector, or a transposon based vector or synthetic mRNA.

The vector may be capable of transfecting or transducing a cell.

### Cell

The present invention provides a cell which expresses a chimeric antigen receptor (CAR) and a mutant version of calcineurin A and/or calcineurin B which is resistant to a calcineurin inhibitor.

The cell may comprise a nucleic acid sequence, a nucleic acid construct or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface.

These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

### Method of Making Cell

The present invention provides a method for making a cell, which comprises the step of introducing: a nucleic acid construct, a kit of nucleic acid sequences, a vector or a kit of vectors of the invention, into the cell. The nucleic acid may be introduced to the cell ex vivo or in vitro.

The method may comprise the following steps:
(i) obtaining a population of cells;
(ii) transducing or transfecting the cells a nucleic acid construct, a kit of nucleic acid sequences, a vector or a kit of vectors of the invention;
(iii) adding a calcineurin inhibitor to the cell population from step (ii).

The presence of the calcineurin inhibitor in step (iii) has the effect of enriching for transduced/transfected cells as cells which express the calcineurin mutant will be resistant to inhibition of proliferation by the calcineurin inhibitor, whereas untransduced cells will be susceptible to such inhibition

The calcineurin inhibitor may, for example, be cyclosporine or tacrolimus.

Cells according to the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo cell from a subject. The cell may be from a peripheral blood mononuclear cell (PBMC) sample. The cells may be activated and/or expanded prior to being transduced with nucleic acid encoding the molecules providing the chimeric polypeptide according to the first aspect of the invention, for example by treatment with an anti-CD3 monoclonal antibody.

### Pharmaceutical Composition

The present invention also relates to a pharmaceutical composition comprising a plurality of cells according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

The pharmaceutical composition may also comprise a calcineurin inhibitor, such as cyclosporine A or tacrolimus.

### Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The cell-containing sample may be isolated from a subject or from other sources, as described above.

The plurality of cells may be administered in the form of a pharmaceutical composition. The pharmaceutical composition may additionally comprise a pharmaceutically acceptable carrier, diluent or excipient. The pharmaceutical composition may optionally comprise one or more further pharmaceutically active polypeptides and/or compounds, for example one or more CNIs as described above. Such a formulation may, for example, be in a form suitable for intravenous infusion.

The present invention provides a cell according to the present invention for use in treating and/or preventing a disease.

The present invention also relates to the use of a cell according to the present invention for the manufacture of a medicament for the treatment and/or prevention of a disease.

The disease to be treated and/or prevented by the method of the present invention may be cancer.

The disease to be treated by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaema (T-ALL) or diffuse large B-cell lymphoma (DLBCL).

The disease may be a plasma cell disorder such as plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance or smoldering multiple myeloma.

The cell of the present invention may be capable of killing target cells, such as cancer cells. The target cell may be recognisable by expression of a TAA, for example the expression of a TAA provided above in Table 1. The cancer may be a cancer listed in Table 1.

The disease may be a transplant-associated cancer, such as post-transplant lymphoproliferative disorder (PTLD) or a melanoma.

The cell of the present invention may be capable of killing a cell implicated in the pathogenesis of an autoimmune disease. Such a cell may be a B-cell which secretes auto-antibodies.

The autoimmune disease may be selected from rheumatoid arthritis, systemic lupus erythematosus, Sjögren's Syndrome, psoriasis, Experimental Autoimmune Encephalomyelitis (EAE), uveitis, pyoderma gangrenosum, membranous nephopathy, atopic dermatitis, Bechet's disease, refractory ulcerative colitis and chronic autoimmune urticara.

The administration of a cell according to the present invention can be accomplished using any of a variety of routes, such as intraperitoneally, intravenously, subcutaneously, transcutaneously or intramuscularly.

Post-Treatment with Calcineurin Inhibitor

The present invention provides a method which comprises the following steps:
(i) administering a plurality of cells of the invention to a subject; and
(ii) subsequently administering a calcineurin inhibitor to the subject.

Step (ii) may involve regular administration of the calcineurin inhibitor to the subject for a period of about 6 weeks following administration of the cells.

The calcineurin inhibitor may be selected from cyclosporine A (CsA), and tacrolimus. The calcineurin inhibitor may be administered orally to the subject.

Tacrolimus is widely used for the prevention of transplant rejection

Treatment of Subject Who is Receiving or has Received Treatment with a Calcineurin Inhibitor

The present invention also provides a method for treating a disease in a subject who is receiving or has received treatment with a calcineurin inhibitor, which method comprises the step of administering to the subject a plurality of cells which express:
(a) a chimeric antigen receptor (CAR); and
(b) a mutant version of calcineurin A and/or calcineurin B which is resistant to the calcineurin inhibitor.

The subject may, for example, be receiving or have received treatment with a calcineurin inhibitor in order to treat and/or prevent an autoimmune disease or to prevent transplant rejection. Such a subject would normally be unsuitable for treatment with CAR-T cells, but may be treated with the cells of the invention which are resistant to immunosuppression with calcineurin inhibitors.

The patient may have recently received a transplanted organ, such as a kidney, heart, liver or other organ. Tacrolimus is routinely given for the prevention of rejection of a transplanted organ and is available is various formulations including capsules (Adoport®, Prograf®, Capexion®, Tacni® and Vivadex®); modified-release capsules (Advagraf®); Granules (Modigraf®): and liquid medicine.

Tacrolimus capsules are usually given twice each day, once in the morning and once in the evening; whereas modified-release capsules are given once each day. The starting dose for capsules is usually between 0.05-0.2 mg/kg/day of body weight for adults (divided into 2 doses and taken every 12 hours) and 0.15-0.3 mg/kg/day of body weight for children (again, divided into 2 doses and taken every 12 hours). For extended release capsules, for adults the dose is usually between 0.15 to 0.2 mg/kg of body weight once a day before reperfusion (return of blood flow to the blocked organ) or within 48 hours after transplant. For children, the dose may be 0.3 mg/kg of body weight given once a day within 24 hours of reperfusion.

Cyclosporine is available as an oral capsule, an oral solution and an injectable form.

Cyclosporine oral capsule is used to treat inflammation in rheumatoid arthritis and psoriasis and also used to prevent the rejection of an organ transplant. There are various brand-name oral capsule versions of cyclosporine, including Gengraf™, Neoral™, and Sandimmune™.

For the treatment of autoimmune diseases such as rheumatoid arthritis and Psoriasis, cyclosporine may be taken at a dosage of between 2.5 to 4 mg/kg/day divided into two doses.

For the prevention of transplant rejection, cyclosporine may be given at 10-15 mg/kg per day, with the first dose taken 4-12 hours following transplant. The same dosage may be taken for 1-2 weeks following transplant and then reduced by 5-10% to a maintenance dosage of 5-10 mg/kg/day.

The subject may have received treatment with a calcineurin inhibitor for days, weeks, months or even years prior to treatment with the CAR-expressing cells of the invention.

### Pre-Conditioning Method

The present invention also provides a method which comprises the following steps:
(i) preconditioning a patient with a calcineurin inhibitor; and
(ii) administering to the subject a plurality of cells which express:
(a) a chimeric antigen receptor (CAR); and
(b) a mutant version of calcineurin A and/or calcineurin B which is resistant to the calcineurin inhibitor.

The patient may be pre-conditioned with a composition or regimen which includes one or more other agents, such as cyclophosphamide. Cyclophosphamide may, for example, be given at a dose of 300-500 mg/m² for 2 or 3 days, ending 2 to 4 days before administration of CAR-expressing cells.

The preconditioning composition or regimen may lack fludarabine.

### Allogeneic Methods

The methods of the present invention are particularly suited to treatment approaches using allogeneic cells.

To date, most CAR therapies have been developed using autologous cells. Although this approach has advantages in terms of immunogenicity, it is associated with several draw-backs. Cell therapy using autologous cells is a labour-intensive process with multiple open operations, leading to a very high cost of goods (COGs). Once a patient is identified, autologous therapies require patient cells to be collected. This is followed by the manufacturing process, which can often take between 2-4 weeks in the case of CAR-T, before the therapy can be provided to the patient. Patient cell performance in the manufacturing process can also vary significantly, leading to batch failures and therapy unavailability for the patient.

These challenges have led to a strong emphasis on the development of allogeneic CAR-T therapies, developed from cells of healthy donors, that could be available for patients immediately in a manner similar to other ‘off’-the-shelf drugs. Allogeneic therapies could be envisioned to serve both as the primary therapy for a disease, once the desired patient efficacy is achieved, or as an additional line of treatment before they are ready for an autologous therapy, if efficacy in certain cases is limited.

By their nature, allogeneic CAR-T therapies enable traditional economies of scale—compared to scale out for autologous therapies—through expansion of batch sizes and increased productivity, making it possible to reach a larger patient population sooner. Clinical trials of allogeneic CAR-T therapies are continuing to increase with approximately 20 therapies currently in the early stages of development.

Rejection of allogeneic cells can be due to minor mismatch or major mismatch both of which are caused by HLA interacting with TCR. In the same way as calcineurin inhibitors may be used to prevent rejection of a transplanted organ, calcineurin inhibitors such as cyclosporine and tacrolimus may be used to prevent rejection of allogeneic CAR-expressing cells by alloreactive host T-cells.

As the CAR-expressing cells of the present invention express mutant calcineurin, the allogeneic CAR-T cells will be resistant to immunosuppression by treatment of the patient with e.g. tacrolimus whereas the alloreactive host T-cells will be inhibited.

Alloreactive responses are a particular problem for CAR-type therapies which involve multiple rounds of administration of CAR-T cells presumably due to triggering of a memory immune response. The method of the invention provides an approach to avoid stimulation of such an immune response by selective immunosuppression of the host T-cells, precluding the generation of such a response.

The method of the invention may involve administering a plurality of doses of the CAR-expressing cells to a subject, in particular, it may involve administration of a plurality of doses of allogeneic CAR-expressing cells.

The invention will now be further described by way of Examples, which are meant to serve to assist one of ordinary skill in the art in carrying out the invention and are not intended in any way to limit the scope of the invention.

### EXAMPLES

### Example 1—Expression of a Calcineurin Mutant by CAR-T Cells Gives Resistance to Inhibition of Proliferation by a Calcineurin Inhibitor

PBMCs were transduced with a vector expressing a CAR together with the sort-suicide gene RQR8 which is described in WO2013/153391. The CARs tested are summarised below:

CD19 CAR: A second generation CAR having an antigen binding domain derived from Fmc63, a hinge spacer and a 41BB/CD3z endodomain

TRBC1 CAR: A second generation CAR having an antigen binding domain as described in WO2018/224844, a hinge spacer and a 41BB/CD3z endodomain

TRBC2 CAR: A second generation CAR having an antigen binding domain as described in WO2020/089644, a CD8 stalk spacer and a CD28/CD3z endodomain

One population of cells were transduced with a tricistronic vector expressing RQR8, the TRBC2 CAR and the CnB30 calcineurin mutant module described above having SEQ ID No. 32.

Transduced cells were co-cultured with one of the following target cell types:
Jurkat TRBC1: wild-type Jurkat cells which express TRBC1
Jurkat KO: Jurkat cells engineered to lack TRBC1 expression
Jurkat TRBC2: Jurkat cells in which the TRBC1 gene is replaced by the TRBC2 gene using CRISPR-Cas9 technology so that the expression of TRBC2 is the same as that of TRBC1 on the wild-type cell.

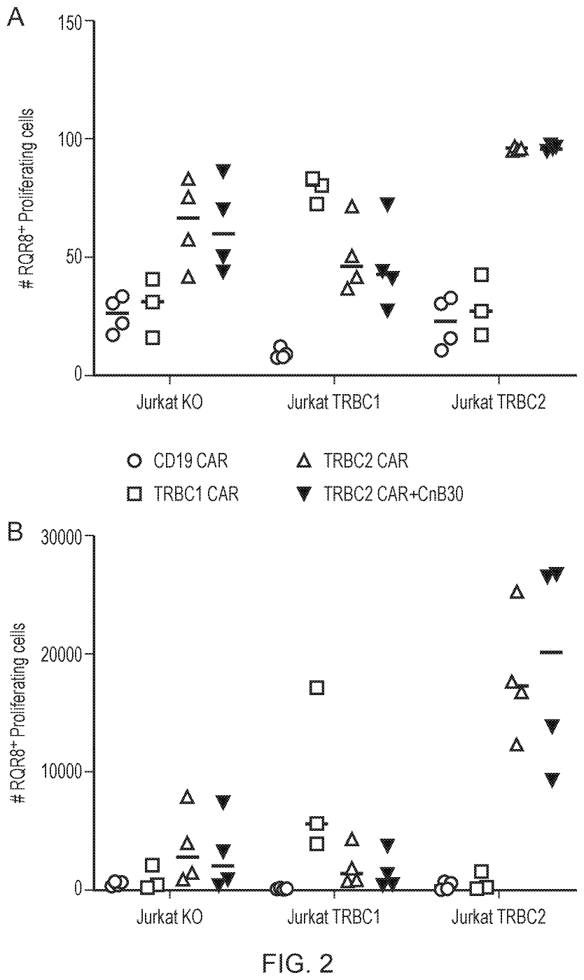

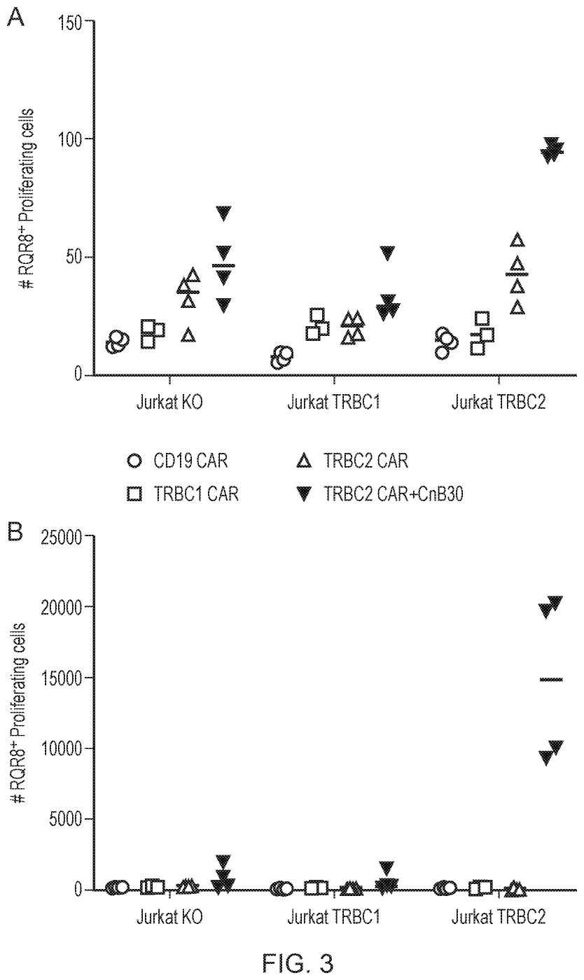

Cells were co-cultured for 96 hours at a 1:4 E:T ratio in the presence or absence of ng/ml of Tacrolimus. Transduced effector cells were identified based on their expression of RQR8 and their proliferation analysed using cell-trace violet (CTV) dilution. The results are shown in FIGS. 2 and 3. As expected, in the absence of Tacrolimus, cells expressing the TRBC1 CAR showed an increase in the percentage and number of proliferating cells following co-culture with TRBC1-expressing target cells; and cells expressing the TRBC2 CAR showed an increase in the percentage and number of proliferating cells following co-culture with TRBC2-expressing target cells (FIG. 2). In the presence of Tacrolimus, proliferation of CAR-T cells is inhibited as can be seen by comparing “TRBC2 CAR” in FIG. 2B (without Tacrolimus) and in FIG. 3B (with Tacrolimus). Only the cells co-expressing the TRBC2 CAR and the CnB30 calcineurin mutant showed an increase in the absolute number (FIG. 3B) of transduced effector cells following co-culture with TRBC2-expressing targets. This population also showed the highest percentage of transduced proliferating cells (FIG. 3A).

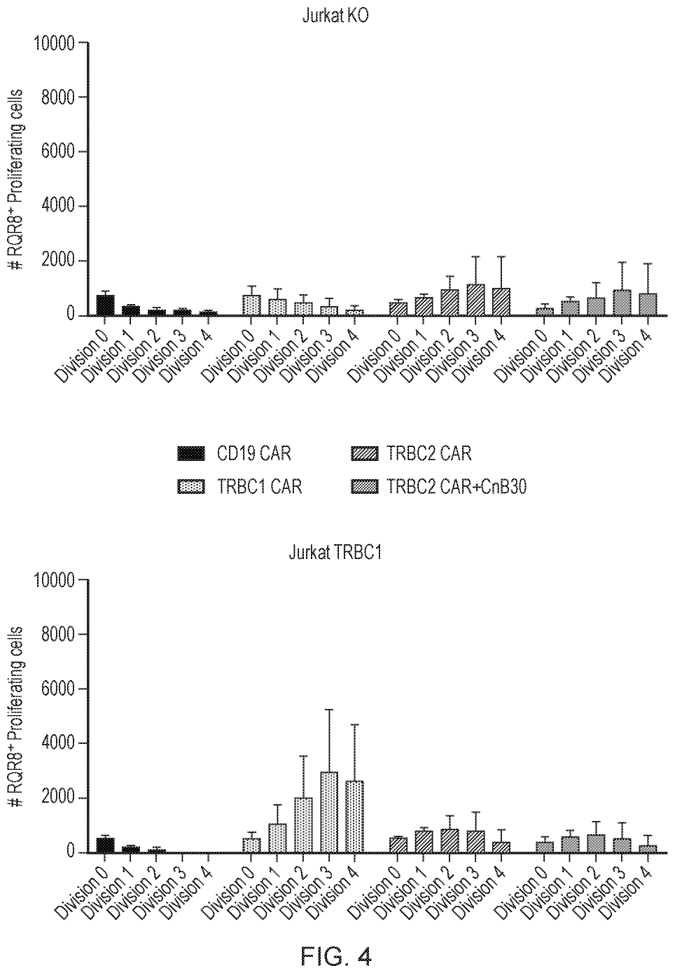

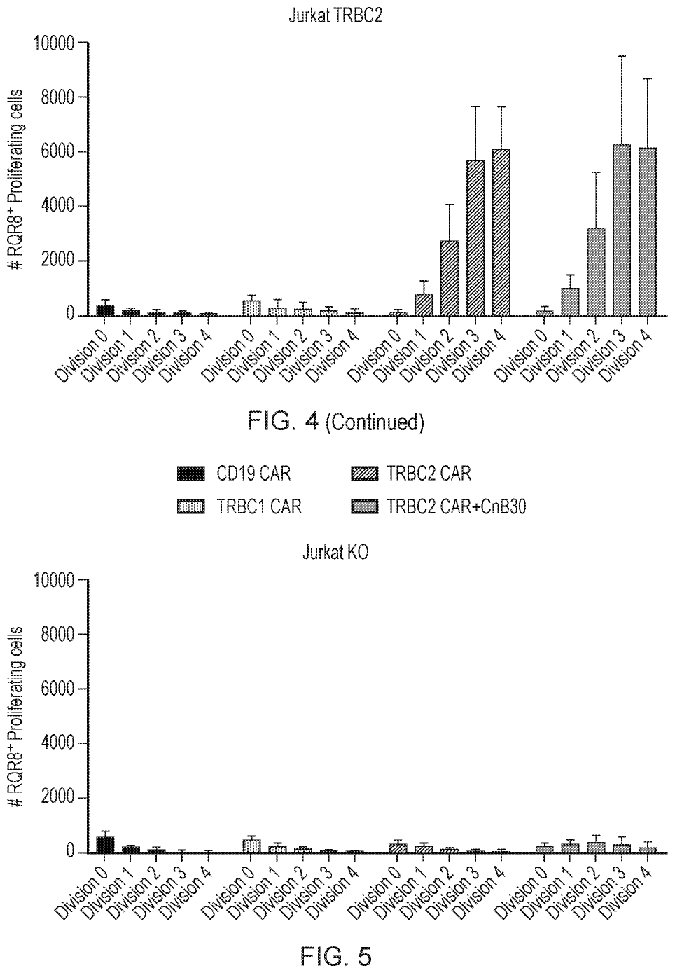

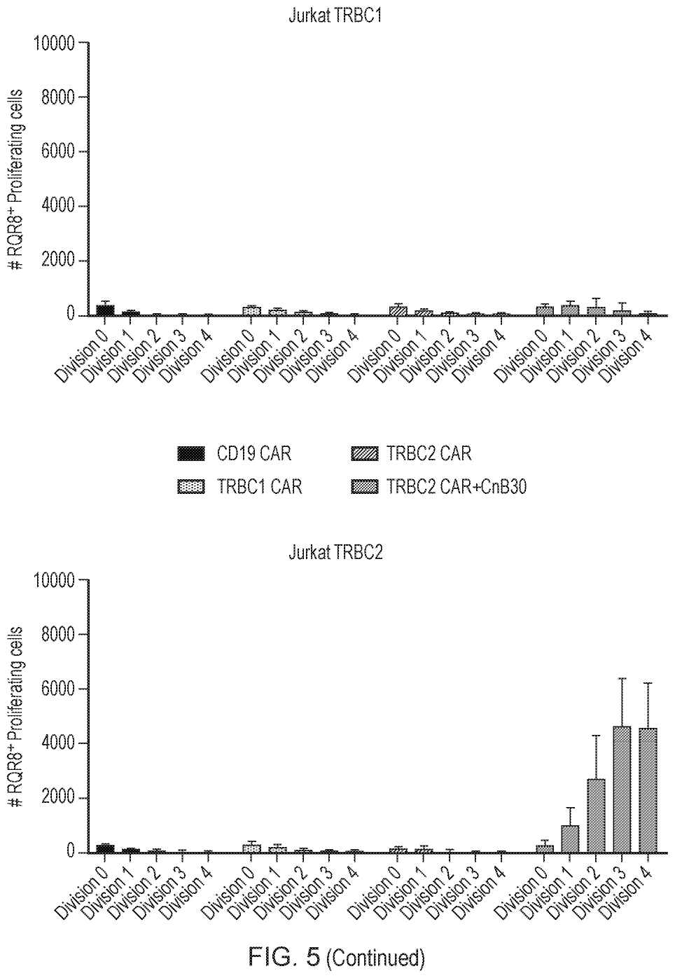

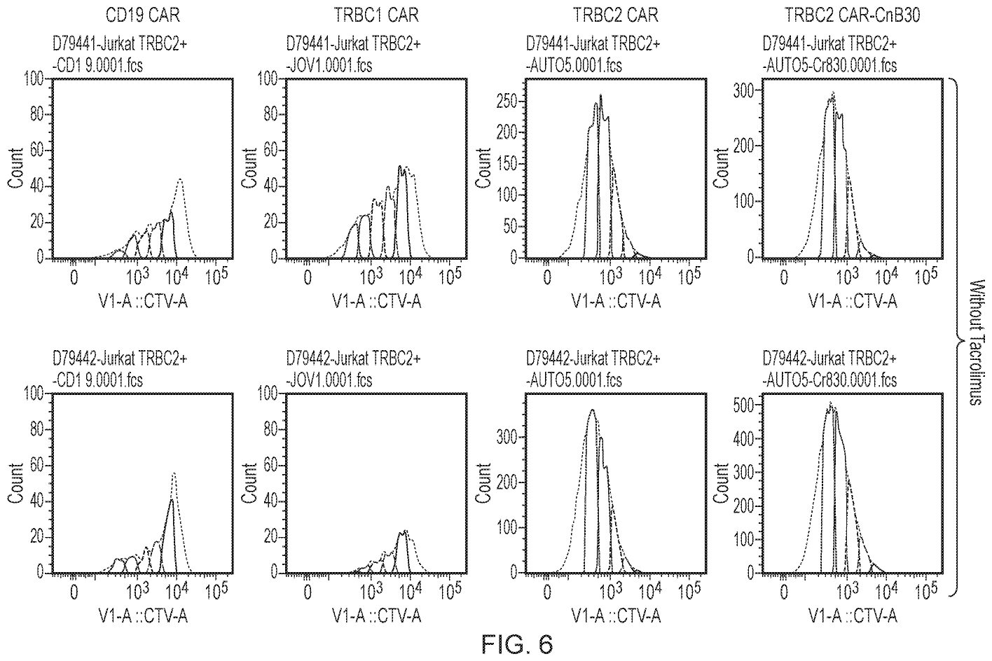

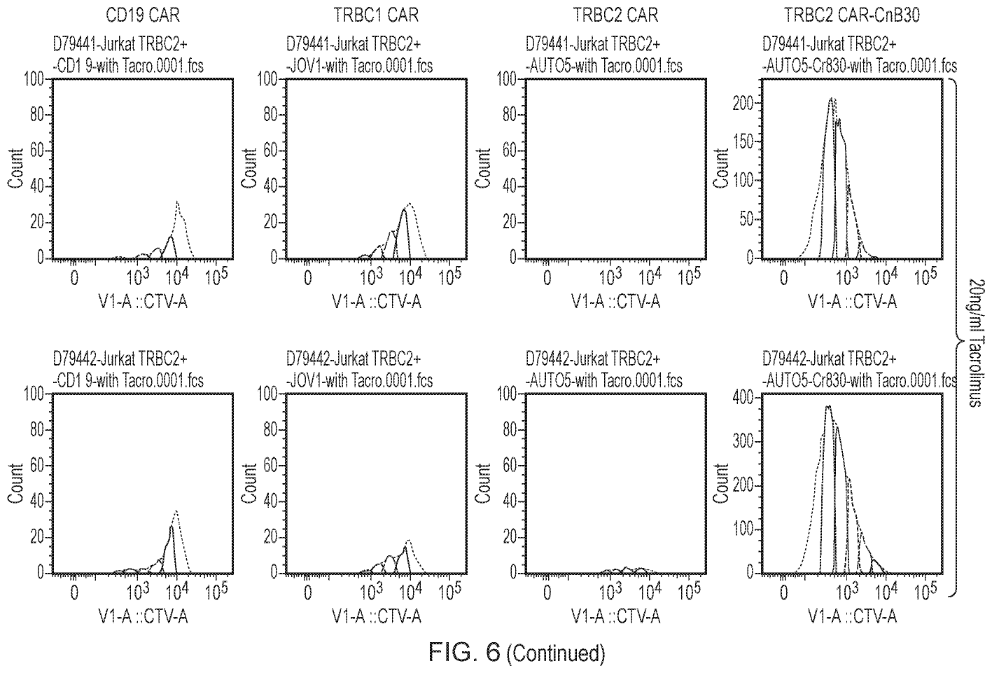

Proliferation analysis was also calculated on single/live/CellTrace Violet-positive cells using the FlowJo proliferation tool using CD19 CAR as the negative control. The cell number in each division was plotted for each CAR+target combination described above and the results are shown in FIGS. 4 (without Tacrolimus) and 5 (with tacrolimus). The results for two separate donors are also shown in the histogram plots of FIG. 6. Again, in the presence of Tacrolimus, only the cells co-expressing the TRBC2 CAR and the CnB30 calcineurin mutant showed an increase in proliferation of effector cells following co-culture with TRBC2-expressing targets (FIG. 5, bottom graph; and FIG. 6).

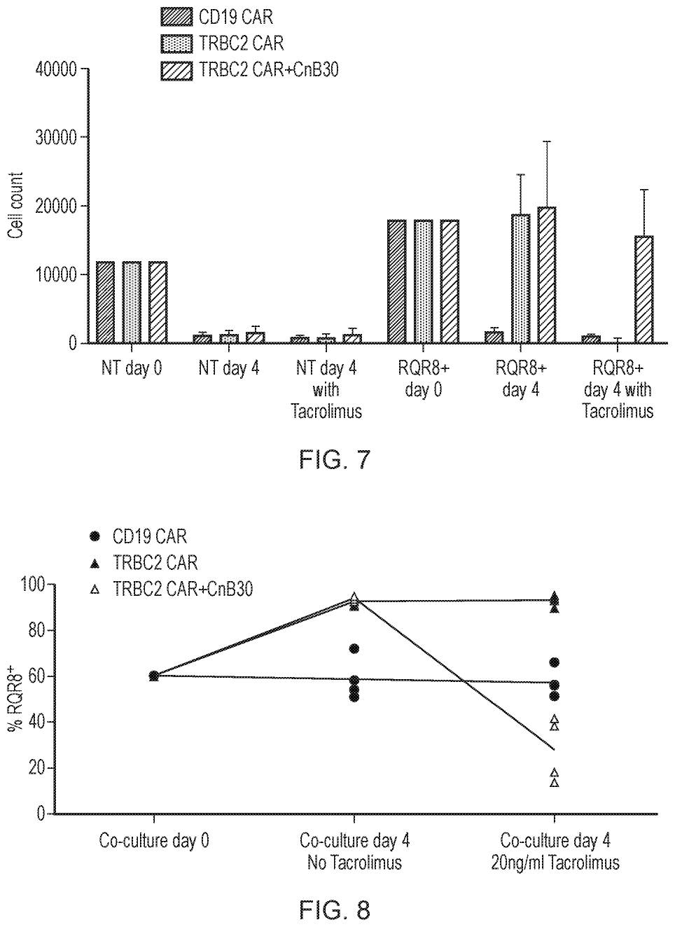

In a similar study, cells transduced to express either TRBC2 CAR alone or TRBC2 CAR in combination with a calcineurin mutant (CnB30) were co-cultured for 4 days with TRBC2+ positive targets in the presence or absence of 20 ng/ml of Tacrolimus. The number CAR-expressing cells after 4 days' co-culture is shown in FIG. 7. The only cell population with a high number of cells expressing TRBC2 CAR after co-culture in the presence of Tacrolimus were the cells co-expressing TRBC2 CAR with the calcineurin mutant (TRBC2 CAR+CnB30).

FIG. 8 shows the percentage of RQR8-expressing cells. While the percentage of CD19-expressing cells stayed constant, the percentage of cells expressing TRBC2 CAR increased following co-culture with TRBC2+ targets in the absence of Tacrolimus. This was true for cells expressing TRBC2 CAR alone or co-expressing TRBC2 CAR in combination with the calcineurin mutant. In the presence of Tacrolimus, the percentage of RQR8+ cells expressing TRBC2 CAR alone was reduced, showing that Tacrolimus inhibits proliferation of these cells. By contrast, the percentage of RQR8+ cells co-expressing TRBC2 CAR/CnB30 was the same as in the co-culture without Tacrolimus, indicating that these cells show resistance to calcineurin inhibition.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for treating a disease in a subject who is receiving or has received treatment with a calcineurin inhibitor, which method comprises the step of administering to the subject a plurality of cells which express:
(a) a chimeric antigen receptor (CAR); and
(b) a mutant version of calcineurin A and/or calcineurin B which is resistant to the calcineurin inhibitor.

2. A method according to claim 1, wherein the subject is receiving or has received treatment with a calcineurin inhibitor in order to treat and/or prevent an autoimmune disease.

3. A method according to claim 2, wherein the CAR binds an autoantigen associated with the autoimmune disease.

4. A method according to claim 1, wherein the subject is post-transplant and is receiving or has received treatment with a calcineurin inhibitor in order to prevent transplant rejection.

5. A method according to claim 4, wherein CAR binds a cancer antigen and method is to treat and/or prevent a transplant-associated cancer.

6. A method according to claim 5, wherein the transplant-associated cancer is a post-transplant lymphoproliferative disorder (PTLD) or a melanoma.

7. A method for treating a disease in a subject, which comprises the following steps:
(i) preconditioning a patient with a calcineurin inhibitor; and
(ii) administering to the subject a plurality of cells which express:
(a) a chimeric antigen receptor (CAR); and
(b) a mutant version of calcineurin A and/or calcineurin B which is resistant to the calcineurin inhibitor.

8. A method according to claim 1 wherein the CAR-expressing cells are allogeneic.

9. A method according to claim 1 which involves administering a plurality of doses of the CAR-expressing cells to the subject.

10. A method according to claim 1, wherein the calcineurin inhibitor is selected from cyclosporine A (CsA), and tacrolimus.

11-13. (canceled)

14. A method according to claim 1, wherein the cells express one of the following mutant versions of calcineurin A and/or calcineurin B:
calcineurin A comprising mutations T351E and L354A with reference to the shown as SEQ ID No. 30;
calcineurin A comprising mutations V314R and Y341F and with reference to shown as SEQ ID No 30; or
calcineurin B comprising mutation L124T and K-125-LA-Ins with reference to shown as SEQ ID No. 31.

15. A method according to claim 14, wherein the cells express mutant calcineurin B having the sequence shown as SEQ ID No. 32.

16. A method according to claim 7 wherein the CAR-expressing cells are allogeneic.

17. A method according to claim 7 comprising administering a plurality of doses of the CAR-expressing cells to the subject.

18. A method according to claim 7, wherein the calcineurin inhibitor is selected from cyclosporine A (CsA), and tacrolimus.

19. A method according to claim 7, wherein the cells express one of the following mutant versions of calcineurin A and/or calcineurin B:
calcineurin A comprising mutations T351E and L354A with reference to the shown as SEQ ID No. 30;
calcineurin A comprising mutations V314R and Y341F with reference to shown as SEQ ID No 30; or
calcineurin B comprising mutation L124T and K-125-LA-Ins with reference to shown as SEQ ID No. 31.

20. A method according to claim 19, wherein the cells express mutant calcineurin B having the sequence shown as SEQ ID No. 32.
